(19) United States
(12) Patent Application Publication
(10) Pub. No.: US 2022/0023372 A1
(43) Pub. Date: Jan. 27, 2022

(54) USE OF COCCULUS HIRSUTUS EXTRACT FOR TREATING DENGUE

(71) Applicants: SUN PHARMACEUTICAL INDUSTRIES LIMITED, Mumbai, Maharashtra (IN); lNTERNATIONAL CENTRE FOR GENETIC ENGINEERING AND BIOTECHNOLOGY, New Delhi, Delhi (IN); DEPARTMENT OF BIOTECHNOLOGY, New Delhi, Delhi (IN)

(72) Inventors: Kaushal NAYYAR, Gurugram (IN); Upasana ARORA, Gurugram (IN); Srinivas PALLA, Gurugram (IN); Mohan PRASAD, Gurugram (IN); Saravanan A, Gurugram (IN); Sumit MADAN, Gurugram (IN); Ruchi SOOD, Gurugram (IN); Navin KHANNA, New Delhi (IN); Ankur PODDAR, New Delhi (IN); Rahul SHUKLA, New Delhi (IN); Mudgal KOTHEKAR, Mumbai (IN)

(21) Appl. No.: 17/311,161

(22) PCT Filed: Dec. 9, 2019
(86) PCT No.: PCT/IB2019/060565

§ 371 Date: Jun. 4, 2021

(30) Foreign Application Priority Data
Dec. 7, 2018 (IN) 201821046412

(51) Int. Cl. A61K 36/59; A61K 31/473; A61K 9/20; A61P 31/14

(52) U.S. Cl. CPC A61K 36/59; A61K 31/473; A61K 9/2009; A61K 2236/333; A61K 9/2013; A61P 31/14; A61K 9/2054

(57) ABSTRACT

The present invention relates to a composite extract of Cocculus hirsutus for use in the prevention and treatment of dengue and its pharmaceutical compositions. Further, it provides for a stable pharmaceutical composition comprising a therapeutically effective amount of the said extract for use in the treatment of dengue virus infection in a mammal. It also provides for a method for reducing viral load in the treatment of dengue virus infection by administering the composite extract or its pharmaceutical composition to a mammal in need thereof.

### FIELD OF THE INVENTION

The present invention relates to a composite extract of Cocculus hirsutus for use in the prevention and treatment of dengue virus infection and its pharmaceutical compositions. It also relates to processes for the preparation of these extracts.

### BACKGROUND OF THE INVENTION

Dengue disease remains a major public health concern around the world. The incidence of dengue has grown dramatically around the world in recent decades. Dengue occurs in tropical and sub-tropical climates worldwide, mostly in urban and semi-urban areas. Severe dengue is a leading cause of serious illness and death among children in many Asian and Latin American countries. According to World Health Organization (WHO) estimates, ˜2.5 billion people around the globe are at risk of dengue, with ˜50 million infections worldwide annually. Dengue spreads to humans through Aedes mosquitoes, which serve as carriers of the disease-causing viruses. There are four serotypes of dengue viruses (DENV-1, -2, -3, and -4) belonging to the family Flaviviridae. Recovery from infection by one serotype of dengue virus provides lifelong immunity against that particular serotype. However, cross-immunity to the other serotypes after recovery is only partial and temporary. Subsequent infections by other serotypes increases the risk of developing severe dengue. Infection with DENVs may be asymptomatic, or may result in a range of clinical symptoms from mild dengue fever (DF) to severe and potentially fatal dengue haemorrhagic fever (DHF) and dengue shock syndrome (DSS). The clinical symptoms for mild dengue fever include high fever, severe headache, pain behind the eyes, muscle and joint pains, nausea, vomiting, swollen glands, and rash. Symptoms usually last for 2-7 days, after an incubation period of 4-10 days following the bite from an infected mosquito. The clinical symptoms for severe dengue appear due to plasma leaking, fluid accumulation, respiratory distress, severe bleeding, or organ impairment. Despite the alarming impact on both human health and the global economy, there is no specific treatment of dengue yet available. Though some live attenuated dengue vaccines are being developed, the challenges faced in dengue vaccine development remain high. Right now, in order to resist this internationally re-emerging public health concern there is neither a specific anti-dengue preventive vaccine available nor an efficacious approved therapy. The occurrence of several DENV serotypes in the same vicinity poses a significant risk to local residents (Taylor-Robinson, J Clin Diag Treat. 2018; 1(2): 50-52).

Thus, there exists an urgent need for an effective dengue treatment that can shorten the duration of the illness, reduce the severity of common symptoms, prevent the development of severe complications, and is easy to formulate. Furthermore, it is highly desirable to develop a dengue treatment that can reduce the viral load at an early stage, such that it may potentially prevent dengue fever as well as a life-threatening severe form of dengue.

Cocculus hirsutus Linn (Menispermeaceae), commonly known as Jal-Jammi (Chopra et al., 1958) or Broom creeper is found in moderately cool and hot regions of Asia tropical and Africa; particularly in Indian subcontinent (IndianBihar, Gujarat, Orissa, Rajasthan, Tamil Nadu and Pakistan), Western Asia (Iran) and Asia temperate (Saudi Arabia, Yemen). It is a perennial climber and reaches 2 to 3 m above ground. Use of Cocculus hirsutus extract for dengue virus infection is not known in the prior art. Present inventors have found that Cocculus hirsutus extracts help to effectively prevent and treat the dengue viral disease. The present invention fulfills the above unmet need by providing safe and effective, patient-compliant dengue treatment.

### SUMMARY OF THE INVENTION

The present invention provides a composite extract of Cocculus hirsutus for use in the prevention and treatment of dengue virus infection and its pharmaceutical compositions. It also provides for a method for reducing viral load in the treatment of dengue virus infection by administering the composite extract or its pharmaceutical composition to a mammal in need thereof. Further, it provides for a stable pharmaceutical composition comprising a therapeutically effective amount of the said extract for use in the treatment of dengue virus infection in a mammal. It also relates to processes for the preparation of these extracts. It further provides the activity of these extracts against dengue virus in mammals. Further, these extracts were found to be safe for administration to humans and did not show any toxic effects even at relatively high doses.

### DETAILED DESCRIPTION OF THE INVENTION

In a first embodiment, the present invention provides a composite extract of Cocculus hirsutus for the treatment of dengue virus infection.

The term “composite extract” as used herein, refers to the extract obtained from Cocculus hirsutus in any concentration comprising a mixture of constituents and is present in the form of liquid, semisolid, solid, gel, paste, dispersion, solution or distillate. Preferably, the composite extract is in the form of a solid, for example, in the form of a powder. The use of the term “composte extract” is used interchangeably with “extract” in the present specification. The composite extract is extracted using the plant mass of Cocculus hirsutus. More preferably, the composite extract is extracted using the stem of Cocculus hirsutus

In one aspect of the above embodiment, the extract can be in the form of a powder or in the form of liquid. Extract in the form of a liquid may be an alcoholic extract, a hydroalcoholic extract, or an aqueous extract. The extracts of Cocculus hirsutus include (a) the extracts obtained by extraction of plant mass of Cocculus hirsutus with one or more solvents, or (b) the fractions obtained by partitioning of the extracts of step (a) with one or more solvents. In a preferred embodiment, the extracts of Cocculus hirsutus include (a) the extracts obtained by extraction of stem of Cocculus hirsutus with purified water, or (b) the fractions obtained by partitioning of the extracts of step (a) with one or more solvents. In a preferred embodiment, the extract is an aqueous extract. More preferably, the extract is an aqueous extract obtained using the stem of Cocculus hirsutus. The solvents in the extract may be removed completely by evaporation or spray drying to obtain a dried extract. The dried extract may be used to prepare a pharmaceutical composition or can be used as such. It may be lyophilized to form a powder, which can then be filled into a capsule of suitable size or can be made into other pharmaceutical compositions such as tablets.

In another embodiment, the present invention provides a process for the preparation of extracts from Cocculus hirsutus for use in the treatment of dengue virus infection comprising extracting the plant mass of Cocculus hirsutus with one or more solvents, concentrating the extract, and drying the extract, or extracting the plant mass of Cocculus hirsutus with one or more solvents, concentrating the extract, adding water and partitioning the extract with one or more solvents, and drying the extract, or extracting the plant mass of Cocculus hirsutus with one or more solvents, concentrating the extract, extracting the extract with one or more solvents, and drying the extract. In another aspect of the above embodiment, the extraction of the plant mass of Cocculus hirsutus is done at a temperature in the range of about 50° to about 100° C. In another aspect of the above embodiment, the extraction of the plant mass of Cocculus hirsutus is done at a temperature of about 80° to about 85° C. In another aspect of the above embodiment, the extraction of the plant mass of Cocculus hirsutus is done at a temperature of about 60° to 65° C. In another aspect of the above embodiment, the drying of extract of Cocculus hirsutus is done at a temperature in the range of about 40° to about 95° C. In another aspect of the above embodiment, the drying of extract of Cocculus hirsutus is done at a temperature in the range of about 40° to about 45° C. In another aspect of the above embodiment, the drying of extract of Cocculus hirsutus is done at a temperature in the range of about 45° to about 50° C. In another aspect of the above embodiment, the drying of extract of Cocculus hirsutus is done at a temperature in the range of about 55° to about 65° C. In another aspect of the above embodiment, the drying of extract of Cocculus hirsutus is done at a temperature in the range of about 90° to about 95° C. In another aspect of the above embodiment, the drying of extract of Cocculus hirsutus is done at a the reflux temperature. Drying of extract can be done either by vacuum rotary evaporation or vacuum pan drying or spray drying etc. Preferably, the drying of extract is done by spray drying.

The term “alcoholic extract,” as used herein, includes any alcohol-based extract, for example, methanolic, ethanolic, n-propanolic, isopropanolic, n-butanolic, iso-butanolic or t-butanolic extract of Cocculus hirsutus.

The term “hydroalcoholic extract,” as used herein, includes an extract prepared by using a mixture of alcohol and purified water. Examples of alcohols are methanol, ethanol, n-propanol, isopropanol, n-butanol, iso-butanol, and t-butanol. Hydroalcoholic extract may be in the ratio of 95:1 to 1:95 mixture of alcohol and purified water. Preferably, a hydroalcoholic extract may be in the ratio of 95:1 to 1:1 mixture of alcohol and purified water. More preferably, hydroalcoholic extract may be in the ratio of 1:1 mixture of alcohol and purified water

The term “aqueous extract,” as used herein, includes a purified water extract of Cocculus hirsutus.

The solvents for extraction may be, for example, water; alcohols, for example, methanol, ethanol, propanol, isopropanol or butanol; ketones, for example, acetone or methyl isobutyl ketone; esters, for example, methyl acetate or ethyl acetate; halogenated hydrocarbons, for example, chloroform, dichloromethane or ethylene dichloride; petroleum fractions, for example, hexane, petroleum ether or heptane; or mixture(s) thereof.

The solvents for partitioning may be, for example, water; petroleum fractions, for example, hexane, petroleum ether or heptane; halogenated hydrocarbons, for example, chloroform, dichloromethane or ethylene dichloride; esters, for example, ethyl acetate or methyl acetate; ketones, for example, acetone or methyl isobutyl ketone; alcohols, for example, butanol; ethers, for example, diethyl ether; or mixture(s) thereof.

The term “plant mass of Cocculus hirsutus,” as used herein, refers to the whole plant or part of the plant, which includes aerial parts, for example, fruits, flowers, leaves, branches, stem bark, stems, seeds or heartwood, and roots. In a preferred embodiment, the “plant mass of Cocculus hirsutus” refers to stem of Cocculus hirsutus.

In a further embodiment, the present invention provides a composite extract of Cocculus hirsutus for use in the prevention and treatment of dengue virus infection in mammals, wherein the extract exhibits a platelet protective effect. In a preferred embodiment, the present invention provides an aqueous extract obtained using the stem of Cocculus hirsutus for use in the prevention and treatment of dengue virus infection in mammals, wherein the extract exhibits a platelet protective effect.

In another embodiment, the present invention provides a composite extract of Cocculus hirsutus for use in the treatment of dengue virus infection in a mammal, wherein the extract reduces the viral load.

In another embodiment, the present invention provides a composite extract of Cocculus hirsutus to reduce the viral load at an early stage in the treatment of dengue virus infection in mammals, wherein the extract exhibits a platelet protective effect.

The composite extract obtained according to this invention is useful both to be directly administrated to a mammal and to be used in the preparation of a pharmaceutical compositions. The dose of extract may be in the range of approximately 0.01 mg/kg to approximately 1500 mg/kg body weight, particularly in the range of approximately 0.05 mg/kg to approximately 1200 mg/kg body weight, more particularly in the range of approximately 0.1 mg/kg to approximately 500 mg/kg body weight, more particularly in the range of approximately 1 mg/kg to approximately 150 mg/kg body weight. Preferably, the dose of the extract may be in the range of approximately 2 mg/kg to approximately 70 mg/kg body weight. The composite extract or its composition may be administered once, twice, thrice or four times a day.

In one embodiment, the extract of Cocculus hirsutus comprise one or more constituents selected from the group consisting of flavonoids, lignans and alkaloids or combinations thereof. Preferably, the composite extract of Cocculus hirsutus comprises magnoflorine as one of the constituents. More preferably, the composite extract of Cocculus hirsutus comprises magnoflorine in an amount of 0.1% to 1% of the total weight of extract in the composition. In a preferred embodiment, the composite extract of Cocculus hirsutus comprises magnoflorine in an amount of 0.45% of the total weight of extract in the composition. In another aspect of the embodiment, the composite extract of Cocculus hirsutus comprises quercetin as one of the flavanoids.

The term “mammal” herein refers to all mammals including humans. Mammals include, by way of example only, humans, non-human primates, cows, dogs, cats, goats, sheep pigs, rats, mice and rabbits.

In another embodiment, the present invention provides a pharmaceutical composition for use in the treatment of dengue virus infection in mammals comprising a composite extract of Cocculus hirsutus and one or more pharmaceutically acceptable excipients. In one aspect of the embodiment, the present invention provides a stable oral pharmaceutical composition for use in the treatment of dengue virus infection in mammals comprising a composite extract of Cocculus hirsutus and one or more pharmaceutically acceptable excipients.

In a further embodiment, the present invention provides a pharmaceutical composition comprising a composite extract of Cocculus hirsutus and one or more pharmaceutically acceptable excipients to reduce the viral load at an early stage in the treatment of dengue virus infection in mammals, wherein the extract exhibits a platelet protective effect. Preferably, the composition is a stable pharmaceutical composition. More preferably, the composition is a stable oral pharmaceutical composition.

In another embodiment, the present invention provides a stable pharmaceutical composition comprising a therapeutically effective amount of a composite extract of Cocculus hirsutus for use in the treatment of dengue virus infection in a mammal.

In another embodiment, the present invention provides a stable pharmaceutical composition comprising a therapeutically effective amount of a composite extract of Cocculus hirsutus for use in the treatment of dengue virus infection in a mammal, wherein the composition when administered to a mammal in need thereof reduces the viral load.

In another embodiment, the present invention provides a stable pharmaceutical composition comprising a therapeutically effective amount of a composite extract of Cocculus hirsutus for use in the treatment of dengue virus infection in a mammal, wherein the composition when administered to a mammal in need thereof reduces the viral load, wherein the extract further exhibits a platelet protective effect.

A “therapeutically effective amount” as used herein refers to an amount of the extract of the invention sufficient to provide a benefit in the treatment or prevention of dengue viral infection disease, to delay or minimize symptoms associated with the infection, or to cure or ameliorate the infection or cause thereof. In particular, a therapeutically effective amount means an amount sufficient to provide a therapeutic benefit in vivo.

The term “pharmaceutical composition,” as used herein, includes any composition that can effectively deliver the extracts of Cocculus hirsutus to the desired site of action to treat or prevent dengue viral infection. The composition can be delivered by any suitable route of administration, such as oral, nasal, pulmonary, transdermal, or rectal. The pharmaceutical composition includes one or more pharmaceutically acceptable excipients. The oral pharmaceutical composition can be in the form of powder, pellets, granules, spheroids, mini-tablets, caplets, tablets, or capsules. The powder can be in the form of a lyophilized powder filled, with pharmaceutically acceptable excipients, into a capsule of suitable size. Preferably, the pharmaceutical composition is in the form of a tablet. The oral pharmaceutical composition can be present in the form of liquid, including but not limited to solutions, suspensions, emulsions or syrups.

A “stable pharmaceutical composition” as used herein refers to a composition which is stable over extended period of time on storage as assessed from the content of one or more impurities in the composition as described in standard textbooks. The stable pharmaceutical composition of the present invention was found to be stable for atleast 3 months at accelerated condition of 40±2° C./75±5% RH; and for atleast 3 months at long term storage condition of 30±2° C./75±5% RH. The product can be stored at room temperature for a shelf life of 6 months to 2 years. This was surprising as the composition comprises of an extract comprising flavonoids, alkaloids such as magnoflorine, lignans etc as constituents and it was challenging to prepare a stable composition comprising these constituents. The stability of the composition can be assessed by determining some parameters like, assay of one or more marker substance on storage. In a preferable embodiment, the marker is magnoflorine. In addition Loss on drying (LOD) and disintegration time (in tablet composition) upon storage can serve as a measure of stability of composition. For a stable composition the specification of LOD was determined to be NMT 2-5% w/w of the composition. The composition of the present invention when storage for 6 months in an HDPE bottle at accelerated condition of 40° C./75% RH, showed no degradation of Magnoflorine content, LOD was found to be within the range of 2-5% w/w and there was no change in the disintegration time.

The stable pharmaceutical composition of the present invention, further comprises one or more pharmaceutically acceptable excipient. Further, the stable pharmaceutical composition may include a coating. Preferably, the coating is film coating.

The term “pharmaceutically acceptable excipients,” as used herein, includes diluents, binders, disintegrants, lubricants, glidants, polymers, flavoring agents, surfactants, preservatives, antioxidants, buffers, and tonicity modifying agents.

Examples of diluents include microcrystalline cellulose, powdered cellulose, starch, starch pregelatinized, dextrates, lactitol, fructose, sugar compressible, sugar confectioners, dextrose, anhydrous lactose, calcium phosphate-dibasic, calcium phosphate-tribasic, calcium sulfate, and mixtures thereof.

Examples of binders include a water-soluble starch, for example, pregelatinized starch; a polysaccharide, for example, agar, gum acacia, dextrin, sodium alginate, tragacanth gum, xanthan gum, hyaluronic acid, pectin, or sodium chondroitin sulfate; a synthetic polymer, for example, polyvinylpyrrolidone, polyvinyl alcohol, carboxyvinyl polymer, polyacrylic acid-series polymer, polylactic acid, or polyethylene glycol; a cellulose ether, for example, methyl cellulose, ethyl cellulose, carboxymethyl cellulose, hydroxyethyl cellulose, hydroxypropyl cellulose, or hydroxypropyl methyl cellulose; and mixtures thereof.

Examples of disintegrants include calcium carbonate, carboxymethyl cellulose or a salt thereof, for example, croscarmellose sodium, crosslinked povidone, low-substituted hydroxypropyl cellulose, and sodium starch glycolate.

Examples of lubricants/glidants include talc, magnesium stearate, hydrogenated vegetable oils, sodium stearyl fumarate, calcium stearate, colloidal silicon dioxide, Aerosil®, stearic acid, sodium lauryl sulphate, sodium benzoate, polyethylene glycol, hydrogenated castor oil, sucrose esters of fatty acids, microcrystalline wax, yellow beeswax, white beeswax, and mixtures thereof.

Examples of flavoring agents include synthetic flavor oils and flavoring aromatics; natural oils or extracts from plants, leaves, flowers, and fruits; and combinations thereof. These may include cinnamon oil, oil of wintergreen, peppermint oils, bay oil, anise oil, eucalyptus, thyme oil, vanilla, citrus oil, including lemon, orange, lime, and grapefruit, and fruit essences including apple, banana, grape, pear, peach, strawberry, raspberry, cherry, plum, pineapple, and apricot.

Examples of surfactants include anionic surfactants, for example, a sulfonic acid or a salt thereof such as benzenesulfonic acid, dodecylbenzenesulfonic acid, or dodecanesulfonic acid; an alkyl sulfate, for example, sodium dodecyl sulfate or sodium lauryl sulfate; cationic surfactants, for example, a tetraalkylammonium salt such as a tetraalkylammonium halide, benzethonium chloride, benzalkonium chloride, or cetylpyridinium chloride; a nonionic surfactant, for example, a (poly) oxyethylene sorbitan long-chain fatty acid ester such as a polyoxyethylene sorbitan monolaurate, for example, a polysorbate; amphoteric surfactants, for example, a glycin compound such as dodecyl-di-(aminoethyl)glycin, a betaine compound such as betaine or dimethyldodecylcarboxybetaine, and a phosphatidic acid derivative such as lecithin; polymeric surfactants, for example, a polyoxyethylene polyoxypropylene glycol such as Pluronic® or poloxamer; and mixtures thereof.

Examples of buffers include phosphate buffers such as dihydrogen sodium phosphate, citrate buffers such as sodium citrate, meglumine, tri(hydroxymethyl) aminomethane, and mixtures thereof.

Examples of tonicity modifying agents include sodium chloride, mannitol, dextrose, glucose, lactose, sucrose, and mixtures thereof.

Examples of solvents for the preparation of the pharmaceutical composition include water; water miscible organic solvents, for example, isopropyl alcohol or ethanol; dipolar aprotic solvents; methylene chloride; acetone; polyethylene glycol; polyethylene glycol ether; polyethylene glycol derivatives of a mono- or di-glyceride; buffers; organic solvents; and combinations thereof.

In a preferred embodiment, the pharmaceutically acceptable excipients in the composition of the invention includes microcrystalline cellulose, anhydrous lactose, croscarmellose sodium, colloidal silicon dioxide and magnesium stearate.

In another embodiment, the present invention provides a process of preparation of pharmaceutical composition for use in the treatment of dengue virus infection comprising extract from Cocculus hirsutus and one or more pharmaceutically acceptable excipients.

In yet another embodiment, the present invention provides a process of preparation of tablet composition of Cocculus hirsutus extract for use in the treatment of dengue virus infection, the process comprising the steps of:

(i) sifting the extract and blending with pharmaceutically acceptable excipients;
(ii) lubricating the blend obtained from step (i) and compressing into tablets and
(iii) film coating the tablets of step (ii).

In yet another embodiment, the present invention provides a process of preparation of tablet composition of Cocculus hirsutus extract for use in the treatment of dengue virus infection, the process comprising the steps of:

(i) blending the extract with pharmaceutically acceptable excipients;
(ii) granulating the blend of step (i) with a solvent;
(iii) lubricating and compressing the blend into tablets and
(iv) film coating the tablets of step (iii).

In a further embodiment, the present invention provides a process of preparation of tablet composition of Cocculus hirsutus extract for use in the treatment of dengue virus infection, the process comprising the steps of:

(i) blending the extract with pharmaceutically acceptable excipients and compacting the mixture;
(ii) milling the compacts and blending with extragranular excipients;
(iii) lubricating the blend of step (ii) and compressing into tablets and
(iv) film coating the tablets of step (iii).

In one embodiment, the present invention provides a method of treating dengue virus infection in a mammal comprising administering a pharmaceutical composition comprising a administering therapeutic effective amount of a composite extract of Cocculus hirsutus to a mammal in need thereof, wherein the extract reduces the viral load.

In one embodiment, the present invention provides a method of treating dengue virus infection in a mammal comprising administering a composite extract of Cocculus hirsutus to a mammal in need thereof, wherein the extract reduces the viral load.

In yet another embodiment, the present invention provides a method of treating dengue virus infection in a mammal comprising administering a pharmaceutical composition comprising a therapeutic effective amount of a composite extract of Cocculus hirsutus to a mammal in need thereof, wherein the extract exhibits a platelet protective effect. It was found that when administered to winstar rats at a dose of 600 or 1200 mg/kg/day by oral gavage for 14 days a higher platelet count was observed.

In yet another embodiment, the present invention provides a method of treating dengue virus infection in a mammal comprising administering a composite extract of Cocculus hirsutus to the mammals in need thereof, wherein the extract exhibits a platelet protective effect.

In another embodiment, of the present invention provide a method of treating dengue virus infection in a mammal comprising administering a pharmaceutical composition comprising a therapeutic effective amount of an extract of Cocculus hirsutus to a mammal in need thereof, wherein the extract reduces the viral load, and wherein the extract further exhibits a platelet protective effect.

In another embodiment, of the present invention provide a method of treating dengue virus infection in a mammal comprising administering a composite extract of Cocculus hirsutus to a mammal in need thereof, wherein the extract reduces the viral load, and wherein the extract further exhibits a platelet protective effect.

In yet another embodiment, the present invention provides a method for reducing the viral load at an early stage in the treatment of dengue virus infection in mammals comprising administering a pharmaceutical composition comprising a therapeutic effective amount of an extract of Cocculus hirsutus to a mammal in need thereof, wherein the extract exhibits a platelet protective effect.

In yet another embodiment, the present invention provides a method for reducing the viral load at an early stage in the treatment of dengue virus infection in mammals comprising administering a composite extract of Cocculus hirsutus to the mammals in need thereof, wherein the extract exhibits a platelet protective effect.

In yet another embodiment, the present invention provides a method of treating both primary and secondary dengue virus infection in mammals comprising administering a composite extract of Cocculus hirsutus to the mammals in need thereof.

In a further embodiment, the present invention provides an oral composition comprising a composite extract of Cocculus hirsutus, wherein the said composition exhibits an inhibitory activity with IC₅₀ value ranging from about 1 to 100 μg/ml as determined using FACS based assay, against dengue virus selected from DENV-1, DENV-2, DENV-3 and DENV-4. In one aspect of the above embodiment, the said extract and composition is used in the treatment of dengue virus infection caused by serotypes selected from DENV-1, DENV-2, DENV-3 and DENV-4 or combinations thereof. In another aspect of the above mentioned embodiment, the dengue virus infection is caused by serotypes selected from DENV-1 and/or DENV-2 or combinations thereof. In another aspect of the embodiment, the dengue virus infection is caused by serotypes selected from DENV-1 and/or DENV-3 or combinations thereof. In another aspect of the embodiment, the dengue virus infection is caused by serotypes selected from DENV-1 and/or DENV-4 or combinations thereof. In another aspect of the embodiment, the dengue virus infection is caused by serotypes selected from DENV-2 and/or DENV-3 or combinations thereof. In another aspect of the embodiment, the dengue virus infection is caused by serotypes selected from DENV-2 and/or DENV-4 or combinations thereof. In another aspect of the embodiment, the dengue virus infection is caused by serotypes selected from DENV-3 and/or DENV-4 or combinations thereof.

The extract of Cocculus hirsutus is not known in the prior art for its inhibitory activity against different serotypes of dengue virus. When tested in an FACS based Neutralization Test (FNT), which is a known method for testing the efficacy of drug against a given virus species, a composite extract of Cocculus hirsutus was found to be very efficacious in inhibiting DV4 serotype. It is known that recovery from infection by one dengue serotype provides lifelong immunity against that particular serotype. However, cross-immunity to the other serotypes after recovery is only partial and temporary. Subsequent infections (secondary infection) by other serotypes increase the risk of developing severe dengue. Thus, the present compounds provide advantage over treatments known in the art which are effective only against a particular serotype, as it was surprisingly found that the composite extract of Cocculus hirsutus and the composition of the present invention were active against all serotypes dengue virus DENV-1, DENV-2, DENV-3 and DENV-4. The composite extract of Cocculus hirsutus and the composition of the invention, exhibits an inhibitory activity with IC₅₀ value ranging from about 1 to 20 μg/ml as determined using FACS based assay, against all the dengue virus DENV-1, DENV-2, DENV-3 and DENV-4. Thus, the extract and composition were effective against both primary and secondary dengue virus infection. The present inventors has found that the extract and the composition when tested in animal model of primary and secondary dengue infection were found to exhibit protection in both primary and secondary dengue AG129 mouse model.

In another aspect of the embodiment, the said composition comprising quercetin as a constituent exhibits an IC₅₀ value ranging from about 2 to 10 μg/ml against DENV-1. In another aspect of the embodiment, the said composition comprising quercetin exhibits an IC50 value ranging from about 1 to 10 μg/ml against DENV-2. In another aspect of the embodiment, the said composition comprising quercetin exhibits an IC50 value ranging from about 5 to 15 μg/ml against DENV-3. In another aspect of the embodiment the said composition comprising quercetin exhibits an IC50 value ranging from about 5 to 20 μg/ml against DENV-4.

In a further embodiment, the present invention provides a method of preventing vascular leakage in severe dengue infection due to primary or secondary dengue infection in mammals comprising administering a composite extract of Cocculus hirsutus to the mammals in need thereof.

Further, the extract and the composition were found to be effective in preventing vascular leakage in severe dengue infection due to primary or secondary dengue infection. Vascular leakage is a prominent manifestation of severe dengue infections (DHF/DSS). This usually occurs during the critical phase of the infection and levels of cytokines like TNFα and IL6 become elevated. In an AG129 mouse model, intestinal vascular leakage was assessed using Evans blue dye which serves as a marker for albumin extravasation. Briefly, 100 μL of Evans blue dye was injected intravenously into the mice. Two hours post injection, animals were euthanized and extensively perfused with sterile PBS. Vascular permeability in the tissues was determined both visually and quantitatively. Results indicate that mice orally fed with the extract or the composition of the invention exhibited lower vascular leakage (in a dose-dependent manner) as compared to untreated group, which corroborates that the extract to inhibit DENV infection.

In a further embodiment, the present invention provides a method of inhibiting the secretion of cytokines in severe dengue infection in mammals comprising administering a composite extract of Cocculus hirsutus to the mammals in need thereof.

The extract and the composition were also found to be effective in inhibiting the secretion of cytokines in severe dengue infection. Pro-inflammatory cytokines like TNF-α and IFN-γ are produced in excessive amounts which leads to disease progression and vascular leakage. In an in-vivo study, the extract or the composition of the invention inhibited the secretion of cytokines in small intestine. Small intestines of AG129 mice challenged with DENV-2 S221-4G2 Immune Complex (IC) and fed with the extract or the composition of the invention possessed lesser amounts of TNFα and IL-6.

In yet another embodiment, the present invention provides a method of treatment of dengue virus infection in mammals comprising administering a composite extract of Cocculus hirsutus to the mammals in need thereof, wherein the extract is effective in a delayed treatment onset. The extract and composition of the invention when administered after 6 to 12 hours of establishing primary dengue infection in a AG129 mice model, it was found that the delay in treatment did not affect the protective efficacy of the extract or the composition.

In yet another embodiment, the present invention provides a method of prevention of dengue virus infection in mammals comprising administering a composite extract of Cocculus hirsutus to the mammals in need thereof. The extract and the composition of the present invention when administered pre-infection were found to be show protective efficacy against secondary dengue infection when tested in an in-vivo study in AG129 mouse model.

In a further embodiment, the extract and composition of the present invention were found to be safe and didn't show any toxic effect when administered in a therapeutically effective dose to the mammal in need thereof. After repeated oral administration at dose levels of 100, 300, 600 or 1200 mg/kg/day by oral gavage in wistar rats for 14 days the hematological, gross and histological changes were determined. At these doses a higher platelet count was observed. Based on these results, NOAEL in Wistar rats, was established at 300 mg/kg/day. Thus the extract was found to be safe and non-toxic for human use in a dose of 4000 mg/day.

In another embodiment, the extract may be co-administered with one or more additional therapeutic agents. In another embodiment, the composition of the invention may further comprise one or more additional therapeutic agents. The one or more additional therapeutic agents may be selected from anti-pyretic/analgesic compounds such as NSAIDs like paracetamol, other anti-viral drugs etc.

The term “co-administration” herein refers to administration of one or more additional therapeutic agents with the extract to a mammal. The extract and additional therapeutic agents may be in a single pharmaceutical composition, or may be in separate pharmaceutical compositions. Each of the extract or additional therapeutic agents may be administered through the same or different routes of administration. Co-administration encompasses administration in parallel or sequentially.

While the following examples are provided to certain embodiments of the invention, these are not intended to be limiting to the scope of the invention.

### EXAMPLES

### Example 1: Preparation of 95:5 Ethanol: Purified Water Extract of Cocculus hirsutus

The plant mass of Cocculus hirsutus (1 kg) was charged into an extractor at ambient temperature*. A mixture of ethanol and purified water (95:5; 6 L) was added and the reaction mixture was heated at a temperature of 60-65° C. for about 3 hours. The extracted mass was filtered, collected and stored in a container. The extraction and filtration steps were repeated with mixture of ethanol and purified water (95:5; 3 L) twice. The three filtered extracts were combined and concentrated to the maximum possible extent under reduced pressure at a low temperature. The extract was decanted into stainless steel trays, and then dried under vacuum at 45-50° C. till ethanol content is not more than 10000 ppm and moisture content is not more than 5%. The dried extract was cooled to about 20-25° C. and unloaded at controlled humidity (RH NMT 40%).

Yield obtained=90 g to 120 g

### Example 2: Preparation of 1:1 Ethanol:Purified Water Extract of Cocculus hirsutus

The plant mass of Cocculus hirsutus (1 kg) was charged into an extractor at ambient temperature*. A mixture of ethanol and purified water (1:1; 6 L) was added and the reaction mixture was heated at a temperature of 60-65° C. for about 3 hours. The extracted mass was filtered, collected and stored in a container. The extraction and filtration steps were repeated with ethanol and purified water (1:1, 3 L) twice. The three filtered extracts were combined and concentrated to the maximum possible extent under reduced pressure at a low temperature. The extract was decanted into stainless steel trays, and then dried under vacuum at 45-50° C. till ethanol content is not more than 10000 ppm and moisture content is not more than 5%. The dried extract was cooled to about 20-25° C. and unloaded at controlled humidity (RH NMT 40%).

Yield obtained=80 g to 120 g

### Example 3: Preparation of an Aqueous Extract of Cocculus hirsutus

The plant mass (stem) of Cocculus hirsutus (1 kg) was charged into an extractor at ambient temperature*. Purified water (6 L) was added and the reaction mixture was heated at reflux temperature for about 3 hours. The extracted mass was filtered, collected and stored in a container. The extraction and filtration steps were repeated with purified water (3 L) twice. The three filtered extracts were combined and concentrated. These are further dried by Vacuum rotary evaporator or Vacuum pan drier or Spray drier. The dried extract was cooled to about 20-25° C. and unloaded at controlled humidity (RH NMT 40%). *The tem “ambient temperature” as used herein, includes a temperature ranging from about 18° C. to about 25° C.

Yield obtained=70 g to 110 g

### Example 4: Preparation of Tablets from the Extract of Cocculus hirsutus Using Direct Compression Technique

TABLE 1

| | | Quantity in mg/tablet | | | |
|---|---|---|---|---|---|
| S/N | Ingredients | (1) | (2) | (3) | (4) |
| 1 | Extract (from Examples | 1000.0 | 1000.0 | 1000.0 | 1000.0 |
| | 1, 2 or 3) | | | | |
| 2 | Magnesium aluminium | 300.0 | 300.0 | 300.0 | 300.0 |
| | trisilicate | | | | |
| 3 | Lactose monohydrate | 90.0 | — | — | — |
| 4 | Dicalcium Phosphate | — | 90.0 | — | — |
| 5 | Starch Pregelatinised | — | — | 90.0 | — |
| 6 | Calcium silicate | — | — | — | 90.0 |
| 7 | Microcrystalline | 40.0 | 40.0 | 40.0 | 40.0 |
| | cellulose | | | | |
| 8 | Colloidal silicon dioxide | 20.0 | 20.0 | 20.0 | 20.0 |
| 9 | Croscarmellose | 40.0 | 40.0 | 40.0 | 40.0 |
| 10 | Magnesium stearate | 10.0 | 10.0 | 10.0 | 10.0 |
| | Core tab wt. | 1500.0 | 1500.0 | 1500.0 | 1500.0 |
| 11 | Opadry Green | 45.0 | 45.0 | 45.0 | 45.0 |
| 12 | Purified water | Qs. | Qs. | Qs. | Qs. |
| | Total weight of coated | 1545.0 | 1545.0 | 1545.0 | 1545.0 |
| | tablet | | | | |

### Manufacturing Procedure:

1. The extract was passed through sieve #10 mesh (2 mm).
2. Step 1 material along with Magnesium aluminium trisilicate was sifted through #14 mesh (1.4 mm).
3. Lactose monohydrate, Dicalcium Phosphate, Starch Pregelatinised and Calcium silicate were sifted through #36 mesh (420μ)
4. Microcrystalline cellulose, Colloidal silicon dioxide and Croscarmellose were passed through #25 mesh (600μ)
5. The material from Steps 3 and 4 was mixed in a blender with step 2 material.
6. The blend obtained from step 5 was lubricated with magnesium stearate and compressed into tablets.
7. Opadry green was dispersed in purified water to prepare a dispersion.
8. The compressed tablets from step 6 were coated with dispersion of step 7.

### Example 5: Preparation of Tablets from the Extract of Cocculus hirsutus Using Wet Granulation Technique (by Rapid Mixer Granulator)

TABLE 2

| | | Quantity in mg/tablet | | | |
|---|---|---|---|---|---|
| S/N | Ingredients | (1) | (2) | (3) | (4) |

Part (a) :Intra granular part

| | | | | | |
|---|---|---|---|---|---|
| 1 | Extract (from Examples | 1000.0 | 1000.0 | 1000.0 | 1000.0 |
| | 1, 2 or 3) | | | | |
| 2 | Magnesium aluminium | 250.0 | 250.0 | 250.0 | 250.0 |
| | trisilicate | | | | |
| 3 | Methanol | Qs. | Qs. | Qs. | Qs. |
| | Total | 1250.0 | 1250.0 | 1250.0 | 1250.0 |

Part (b):Extra granular part

| | | | | | |
|---|---|---|---|---|---|
| 4 | Magnesium aluminium | 40.0 | 40.0 | 40.0 | 40.0 |
| | trisilicate | | | | |
| 5 | Lactose monohydrate | 90.0 | — | — | — |
| 6 | Dicalcium Phosphate | — | 90.0 | — | — |
| 7 | Starch Pregelatinised | — | — | 90.0 | — |
| 8 | Calcium silicate | — | — | — | 90.0 |
| 9 | Microcrystalline | 40.0 | 40.0 | 40.0 | 40.0 |
| | cellulose | | | | |
| 10 | Colloidal silicon dioxide | 20.0 | 20.0 | 20.0 | 20.0 |
| 11 | Croscarmellose | 40.0 | 40.0 | 40.0 | 40.0 |
| 12 | Magnesium Stearate | 10.0 | 10.0 | 10.0 | 10.0 |
| | Core tab wt. | 1490.0 | 1490.0 | 1490.0 | 1490.0 |
| 13 | Opadry Green | 44.7.0 | 44.7 | 44.7 | 44.7 |
| 14 | Purified water | Qs. | Qs. | Qs. | Qs. |
| | Total weight of coated | 1534.7 | 1534.7 | 1534.7 | 1534.7 |
| | tablet | | | | |

### Manufacturing Procedure:

1. The extract was passed through sieve #10 mesh (2 mm).
2. Magnesium aluminium trisilicate was sifted through sieve #36 mesh (420μ)
3. The material from Step 1 and step 2 was granulated with methanol and dried.
4. The dried material from step 3 was passed through 16 mesh (1 mm)
5. Magnesium aluminium trisilicate, Lactose monohydrate, Dicalcium Phosphate, Starch Pregelatinised and Calcium silicate were sifted through #36 mesh (420μ)
6. Microcrystalline cellulose, Colloidal silicon dioxide and Croscarmellose were passed through #25 mesh (600μ)
7. The material from steps 5 and 6 were mixed in a blender along with step 4 material.
8. The blend obtained from above step was lubricated with magnesium stearate and compressed into tablets.
9. Opadry green was dispersed in purified water to prepare a dispersion.
10. The compressed tablets from step 8 were coated with dispersion of step 9.

### Example 6: Preparation of Tablets from the Extract of Cocculus hirsutus Using Dry Granulation Technique (by Roller Compactor)

TABLE 3

| | | Quantity in mg/tablet | | | |
|---|---|---|---|---|---|
| S/N | Ingredients | (1) | (2) | (3) | (4) |

Part (a): Intra granular part

| | | | | | |
|---|---|---|---|---|---|
| 1 | Extract (from Examples | 1000.0 | 1000.0 | 1000.0 | 1000.0 |
| | 1, 2 or 3) | | | | |
| 2 | Magnesium aluminium | 300.0 | 300.0 | 300.0 | 300.0 |
| | aliminium trisilicate | | | | |
| 3 | Magnesium stearate | 15.0 | 15.0 | 15.0 | 15.0 |
| | Total | 1315.0 | 1315.0 | 1315.0 | 1315.0 |

Part (b) Extra granular part

| | | | | | |
|---|---|---|---|---|---|
| 4 | Lactose monohydrate | 30.0 | — | — | — |
| 5 | Dicalcium Phosphate | — | 30.0 | — | — |
| 6 | Starch Pregelatinised | | — | 30.0 | — |
| 7 | Calcium silicate | | — | — | 30.0 |
| 8 | Microcrystalline | 100.0 | 100.0 | 100.0 | 100.0 |
| | cellulose | | | | |
| 9 | Colloidal silicon dioxide | 10.0 | 10.0 | 10.0 | 10.0 |
| 10 | Croscarmellose | 30.0 | 30.0 | 30.0 | 30.0 |
| 11 | Magnesium stearate | 15.0 | 15.0 | 15.0 | 15.0 |
| | Core tab wt | 1500.0 | 1500.0 | 1500.0 | 1500.0 |
| 12 | Opadry Green | 45.0 | 45.0 | 45.0 | 45.0 |
| 13 | Purified water | Qs. | Qs. | Qs. | Qs. |
| | Total weight of coated | 1545.0 | 1545.0 | 1545.0 | 1545.0 |
| | tablet | | | | |

### Manufacturing Procedure:

1. The extract was passed through #10 mesh (2 mm),
2. The step 1 material was sifted along with Magnesium aluminium trisilicate, through #14 mesh (1.4 mm).
3. Magnesium stearate was sifted through #36 mesh (420μ) and mixed with step 2 material in a blender.
4. The blended material was compacted using roll compactor.
5. The compacts obtained from step 4 were milled.
6. The extra granular excipients were sifted and blended with magnesium stearate to obtain a lubricated blend.
7. The lubricated blend of step 6 was compressed into tablets.
8. Opadry green was dispersed in purified water to prepare a dispersion.
9. The compressed tablets from step 7 were coated with dispersion of step 8.

### Example 7: Preparation of Tablets from the Extract of Cocculus hirsutus

TABLE 4

| | Quantity: % w/w | |
|---|---|---|
| | For tablet | For tablet containing |
| | containing 25 | 100 mg/300 |
| INGREDIENTS | mg extract | mg/500 mg extract |
| Intragranular Part | | |
| Aqueous extract of Cocculus | 16.0 | 64.1 |
| hirsutus* (example 3) | | |
| Microcrystalline cellulose IP ** | 30.0 | 9.0 |
| Anhydrous Lactose IP | 38.5 | 3.9 |
| Croscarmellose sodium IP | 9.0 | 3.9 |
| Colloidal silicon Dioxide IP | 1.3 | 0.6 |
| Magnesium Stearate IP | 1.3 | 0.5 |
| Extra granular Part | | |
| Microcrystalline cellulose IP | — | 7.7 |
| Croscarmellose sodium IP | — | 5.0 |
| Colloidal silicon Dioxide IP | — | 0.6 |
| Magnesium Stearate IP | — | 0.8 |
| Film Coating | | |
| Opadry AMB II Brown | 3.9 | — |
| Opadry AMB II Green | — | 3.9 |
| Purified water IP ## | Q.S. | Q.S. |

*This quantity of Aqueous extract of Cocculus hirsutus is based on 100% w/w of LOD. Actual quantity of API is to be calculated based on actual LOD.
$= \frac{25 / 100 / 300 / 500 \times 100}{( 100 - \% w / w of LOD )} mg$ ** Adjust the quantity of Microcrystalline Cellulose based on the actual quantity of Cocculus hirsutus to keep constant average tablet weight.
## Loss during coating/drying process.

BRIEF MANUFACTURING PROCESS (for 25 mg)
1. Sift Aqueous extract of Cocculus hirsutus, microcrystalline cellulose, anhydrous lactose, croscarmellose sodium and colloidal silicon dioxide through suitable sieve.
2. Blend material of step (1) in blender
3. Sift magnesium stearate through suitable sieve and blend with material of Step (2).
4. Compress the step (3) blend using appropriate tools.
5. Disperse Opadry AMB II brown in purified water to make 20% w/w dispersion and stir well to get uniform dispersion.
6. Load the core tablets into coating pan and coat using coating dispersion to a target weight build-up of approx. 4.0% w/w (3.5-4.5% w/w).

BRIEF MANUFACTURING PROCESS (for 100/300/500 mg)
1. Sift Aqueous extract of Cocculus hirsutus, microcrystalline cellulose, anhydrous lactose, croscarmellose sodium and colloidal silicon dioxide through suitable sieve.
2. Blend material of step (1) in blender.
3. Sift magnesium stearate through suitable sieve and blend with material of Step (2).
4. Compaction/Slugging of the step (3) materials.
5. Mill the compacts/slugs of step (4) through oscillating granulator/multimill.
6. Sift the granules obtained in step (5) through suitable sieve.
7. If required repeat step (4) to step (6) till the desired percentage of granules (NLT 65%) obtained.
8. Sift extra granular materials i.e. microcrystalline cellulose, croscarmellose sodium & colloidal silicon dioxide through suitable sieve.
9. Blend the materials of step (7) and step (8) in a blender.
10. Sift magnesium stearate through suitable sieve and blend with material of step (10).
11. Compress the step (10) blend using appropriate tooling for respective strengths.
12. Disperse Opadry AMB II green in purified water to make 20% w/w dispersion and stir well to get uniform dispersion.
13. Load the core tablets of respective strengths into coating pan and coat using coating dispersion to a target weight buildup of approx. 4.0% w/w (3.5-4.5% w/w).

### Example 8: Stability Study

The stability of the tablets prepared in Example 7 were tested in an accelerated condition of 40° C./75% RH for 6 months.

As seen in Table 5, after storage for 6 months in an HDPE bottle at accelerated condition of 40° C./75% RH, no degradation of Magnoflorine content was observed, LOD was found to be within the range of 2-5% of specification and there was no change in the disintegration time.

TABLE 5
6M Accelerated Stability data for 100/300/500 mg tablets

| Strength | 100 mg | | | | |
|---|---|---|---|---|---|
| Stage | Initial | 1M | 2M | 3M | 6M |
| Magnoflorine content (% w/w) | 0.37 | 0.37 | 0.37 | 0.38 | 0.43 |
| LOD (% w/w) | 4.0 | 2.9 | 2.8 | 6.7 | 2.7 |
| Disintegration time (mins) | 12 | 13 | 13 | 14 | 13 |
| Strength | 300 mg | | | | |
| Magnoflorine content (% w/w) | 0.41 | 0.41 | 0.40 | 0.41 | 0.45 |
| LOD (% w/w) | 3.4 | 2.9 | 2.9 | 6.7 | 3.2 |
| Disintegration time (mins) | 16 | 18 | 18 | 17 | 16 |
| Strength | 500 mg | | | | |
| Magnoflorine content (% w/w) | 0.41 | 0.40 | 0.40 | 0.41 | 0.45 |
| LOD (% w/w) | 3.9 | 3.6 | 3.5 | 5 | 3.8 |
| Disintegration time (mins) | 21 | 24 | 23 | 24 | 24 |

### Example 9: Biological Activity

### FACS-Based Assay

This assay was used to detect the number of DENV-infected cells in total cell population. Vero cells in a 96-well flat bottom plate (sterile, tissue culture treated) Dulbecco modified Eagle medium supplemented with 10% heat inactivated Fetal Bovine Serum AFBS (20,000-25,000 cells/200 μl/well) were seeded and incubated at 37° C. in a humidified incubator with 10% CO2 for 24-26 h (doubling time for Vero cells). In this method, the cells should not be less than 20,000/well. The media was aspirated and cells were infected with DENV-1, 2, 3, 4 at 0.1 MOI in DMEM supplemented with 0.5% AFBS media (100 W/well). The plates were incubated for 2 h at 37° C. in a humidified incubator with 10% CO2. The working stocks of Cocculus hirsutus aqueous extract were prepared and its different concentrations viz. 100 μg/ml, 50 μg/ml, 25 μg/ml, 12.5 μg/ml, 6.25 μg/ml and 3.125 μg/ml to the respective wells of the 96-well plate. The plates were allowed to incubate at 37° C. in a humidified chamber with 10% CO2 for 42-46 h post infection. After completion of the incubation period, cells were stained for the presence of cytosolic DENVs with a fluorescent labelled antibody. For staining, media was aspirated from the top of the cells, washed with PBS. Cells trypsinised and transferred to a 96 well U bottom plate. Then centrifuged and supernatant was aspirated. Cells were washed twice with permeabilization or perm buffer and blocked with 1% normal mouse sera (prepared in perm buffer) for 30 mins. 2H2-Alexa488 antibody was added to stain the cells for DENVs and incubated with gentle shaking. After incubation, cells were centrifuged and supernatant aspirated. Cells were washed and re-suspended in PBS. The above processed cells were analyzed through flow cytometer and 5000 cells were counted per well. Data obtained was analyzed (through FlowJo software) to determine the relative percentage of infected cells for each extract concentration with respect to virus only (without any extract treatment) control group. The IC50 of extract was determined as the extract concentration that inhibited 50% of dengue virus infection, calculated using GraphPad Prism software.

Upon evaluation through FNT, the extract was found to be efficacious in inhibiting all four DENV serotypes. The IC50 values for each DENV serotypes typically ranged between 5-10 μg/ml (Table 6), indicating that 5-10 μg/ml of extract is required to inhibit all four DENV infection by 50% in this assay.

TABLE 6
Tabular representation of IC₅₀ (μg/ml) of the extract against
all the four dengue virus serotypes as examined through FNT.

| | IC₅₀ (μg/ml) | | | |
|---|---|---|---|---|
| Test Candidate | DENV-1 | DENV-2 | DENV-3 | DENV-4 |
| Cocculus hirsutus | 7.3 | 8.2 | 5.5 | 6.9 |
| aqueous extract | | | | |

We claim:

1. A stable pharmaceutical composition comprising a therapeutically effective amount of a composite extract of Cocculus hirsutus for use in the treatment of dengue virus infection in a mammal, wherein the composition when administered to a mammal in need thereof reduces the viral load.

2. The composition of claim 1, wherein said composite extract of Cocculus hirsutus further exhibits a platelet protective effect.

3. The composition of claim 1, wherein said composite extract of Cocculus hirsutus is selected from an alcoholic extract, a hydroalcoholic extract and an aqueous extract.

4. The composition of claim 3, wherein said composite extract of Cocculus hirsutus is an aqueous extract.

5. The composition of claim 4, wherein said aqueous extract of Cocculus hirsutus is extracted using the stem of Cocculus hirsutus.

6. The composition of claim 1, wherein said composition is effective against all dengue virus serotypes DENV-1, DENV-2, DENV-3 and DENV-4.

7. The composition of claim 1, wherein the composition is an oral composition.

8. The composition of claim 7, wherein the composition is stable for atleast 3 months at accelerated condition of 40±2° C./75±5% RH.

9. The composition of claim 1, further comprising one or more pharmaceutically acceptable excipients.

10. The composition of claim 9, wherein the pharmaceutically acceptable excipients are selected from the group comprising diluents, binders, disintegrants, lubricants, glidants, polymers, flavoring agents, surfactants, preservatives, antioxidants, buffers, and tonicity modifying agents.

11. The composition of claim 10, wherein the diluents is selected from microcrystalline cellulose, powdered cellulose, starch, starch pregelatinized, dextrates, lactitol, fructose, sugar compressible, sugar confectioners, dextrose, anhydrous lactose, calcium phosphate-dibasic, calcium phosphate-tribasic, calcium sulfate, and mixtures thereof, and disintegrants is selected from calcium carbonate, carboxymethyl cellulose substituted hydroxypropyl cellulose, and sodium starch glycolate, and lubricants/glidants is selected from talc, magnesium stearate, hydrogenated vegetable oils, sodium stearyl fumarate, calcium stearate, colloidal silicon dioxide, Aerosil®, stearic acid, sodium lauryl sulphate, sodium benzoate, polyethylene glycol, hydrogenated castor oil, sucrose esters of fatty acids, microcrystalline wax, yellow beeswax, white beeswax, and mixtures thereof.

12. The composition of claim 9, wherein the pharmaceutically acceptable excipients comprises microcrystalline cellulose, anhydrous lactose, croscarmellose sodium, colloidal silicon dioxide and magnesium stearate.

13. The composition of claim 1, wherein the composite extract of Cocculus hirsutus comprises magnoflorine as one of the constituents.

14. The composition of claim 13, wherein the composite extract of Cocculus hirsutus comprises magnoflorine in an amount of 0.1% to 1% of the total weight of extract in the composition.

15. The composition of claim 14, wherein the composite extract of Cocculus hirsutus comprises magnoflorine in an amount of 0.45% of the total weight of extract in the composition.

16. A method of treating dengue virus infection in a mammal comprising administering a pharmaceutical composition comprising a therapeutic effective amount of a composite extract of Cocculus hirsutus to a mammal in need thereof, wherein the extract reduces the viral load.

17. The method of claim 16, wherein said composite extract of Cocculus hirsutus further exhibits a platelet protective effect.

18. The method of claim 16, wherein said composite extract of Cocculus hirsutus is selected from an alcoholic extract, a hydroalcoholic extract and an aqueous extract.

19. The method of claim 18, wherein said composite extract of Cocculus hirsutus is an

20. aqueous extract.

21. The composition of claim 19, wherein said aqueous extract of Cocculus hirsutus is extracted using the stem of Cocculus hirsutus.

22. The method of claim 16, wherein said composite extract of Cocculus hirsutus is effective against all dengue virus serotypes DENV-1, DENV-2, DENV-3 and DENV-4.

23. The method of claim 16, wherein the composition reduces the viral load at an early stage in the treatment of dengue virus infection.
